(19) United States
(12) Patent Application Publication
(10) Pub. No.: US 2011/0130421 A1
(43) Pub. Date: Jun. 2, 2011

(54) Methods and Compositions for the Treatment of Chronic Renal Hypertension

(75) Inventors: Christopher S. Wilcox, Washington, DC (US); William J. Welch, Sterling, VA (US); Fredrik Palm, Arlington, VA (US)

(73) Assignee: Georgetown University, Washington, DC (US)

(21) Appl. No.: 12/914,083

(22) Filed: Oct. 28, 2010

Related U.S. Application Data
(60) Provisional application No. 61/256,079, filed on Oct. 29, 2009.

(51) Int. Cl. A61K 31/445; A61P 13/12

(52) U.S. Cl. 514/315

(57) ABSTRACT

Methods and compositions for the treating chronic renal hypertension in a subject comprising administering a therapeutically effective amount of at least one nitroxide-containing composition to the subject, particularly 4-hydroxy-2,2,6,6-tetramethyl-1-piperidine-1-oxyl (Tempol).

### CROSS-REFERENCE TO RELATED APPLICATIONS

Priority is claimed to U.S. Provisional Patent Application No. 61/256,079 filed 29 Oct. 2009, and is hereby incorporated by reference in its entirety.

### BACKGROUND OF THE INVENTION

The invention relates generally to methods and compositions for the treatment of chronic renal hypertension and delaying the onset of renal failure in a subject by administering nitroxides, such as 4-hydroxy-2,2,6,6-tetramethyl-1-piperidine-1-oxyl (Tempol).

Chronic renal hypertension is a secondary form of high blood pressure caused by abnormal changes in the regular blood flow to the kidneys. Chronic renal hypertension can develop as a primary problem, e.g., when there is a stenosis (narrowing) of one of both main arteries to the kidney, or as a secondary problem, for example, in patients who have developed hypertension that later leads to damage of the kidneys.

Patients who have the primary form of renal artery stenosis leading to chronic renal hypertension fall into two main categories. The first, and quantitatively most important, are those who have atherosclerotic narrowing after the origin of one or both renal arteries as they emerge from the aorta. The second category are those patients with fibromuscular dysplasia of the renal artery.

Unfortunately, the former is a progressive condition which, if untreated, can lead to progressive loss of function in the kidney (termed ischemic nephropathy). Reduced renal blood flow results in excessive rennin release and subsequent angiotensin II production which elevates the arterial blood pressure. The reduced renal blood flow together with the elevated circulating angiotensin II levels result in increased oxidative stress, reduced oxygen delivery and relative hypoxia in the affected kidney, which induces irreversible morphological and functional changes in the kidney. The elevated arterial pressure together with the increased oxidative stress also induce structural and functional changes of the heart and arteries, which further contributes to maintaining the hypertension.

Chronic renal hypertension is a condition quite distinct from essential hypertension, a more frequently occurring condition in which high blood pressure develops slowly (as opposed to rather rapidly in chronic renal hypertension) in the absence of any impairment of kidney function or narrowing of the arteries to the kidney. While the causes of essential hypertension are unclear, it involves both genetic predisposition and environmental influences, for example, a high salt intake. Essential hypertension is differentiated from chronic renal hypertension because, by definition, it is not secondary to some other condition such as renal artery disease or kidney insufficiency. A large category of chronic renal hypertension patients are those who have damage to the kidneys that leads to a progressive increase in blood pressure. Since high blood pressure still damages the kidneys, this process can be self-sustaining in the absence of an effective treatment.

The 2K,1C Goldblatt rat is an animal model of renovascular hypertension in humans. In this animal model, hypertension is reversible and the process corrected if the clip or narrowing of the artery to the kidney is corrected soon after the clip is placed. However, over time, the pathophysiology of hypertension changes, as both the unclipped kidney and clipped kidney show pathological changes and diminished function that contributes to the sustained and increasing levels of blood pressure. At this chronic phase, hypertension is not correctable by removing the clip or even removing the clipped kidney. Consequently, the 2K,1C rat model develops more severe hypertension, which quite often leads to premature death due to hypertensive disease and stroke.

The initial acute phase of renovascular hypertension is characterized by being renin-dependent (i.e., renal ischemia releasing renin from the kidney and generating angiotensin II). Up to 2 to 4 weeks after a clip is placed on one renal artery of a rat, hypertension is reversible if the clip is removed or the rat is treated with an angiotensin receptor blocker or an angiotensin-converting enzyme inhibitor.

The chronic phase of renovascular hypertension is characterized by irreversible changes in kidney function, morphology and sustained hypertension. The transition from the acute to chronic stage occurs after 3 or more months post-clipping and is fully established by 9 to 12 months. At this late stage of chronic 2K,1C Goldblatt hypertension, the hypertension and renal insufficiency are not corrected, or even usually improved, by removing the clip or removing the clipped kidney. Therefore, the hypertension is “irreversible.” At this stage, there is damage in both kidneys as a consequence of the high blood pressure and the ischemia. The plasma renin activity and angiotensin II levels, which were initially greatly raised in the blood and kidneys, now return to normal or even below. Consequently, these changes are not corrected by conventional treatments directed against the renin-angiotensin system or surgical correction of the renal artery restriction.

In the 2K,1C Goldblatt rat, clipping of a renal artery reduces renal perfusion pressure, which increases angiotensin II (Ang II) concentrations in both kidneys. Ang II acts through several mechanisms to produce reactive oxygen species (ROS).

ROS play a role in oxidative stress in the kidney. For example, it has been reported that O₂ availability in clipped kidneys of 2K,1C Goldblatt rats in the early stages of renal hypertension is maintained by Ang II acting on Ang II type 2 receptors (AT₂-Rs), resulting in nitric oxide (NO) release. Ang II also activates nicotinamide adenine dinucleotide phosphate (NADPH) oxidase through Ang II type 1 receptors (AT1-Rs), producing superoxide (O₂.⁻). Prolonged Ang II infusion reduces renal tissue pO₂, which is ascribed to excessive formation of ROS.

These ROS play a role in oxidative stress in the kidney, and there is evidence that this oxidative stress plays a role in the early stages of renal hypertension. It has been reported that prolonged administration of Tempol in two-kidney, one-clip (2K,1C) Goldblatt rats in the early stages of renal hypertension reduced mean arterial pressure (MAP), improved renal blood flow, improved the glomerular filtration rate (GFR), and improved oxygen tension (pO₂) of the clipped kidney. Blockade of AT₁-Rs with candesartan also reduced the MAP, but failed to improve the renal hemodynamics or oxygenation.

In stark contrast to what is observed in early stage 2K,1C Goldblatt rats, it has been reported that the administration of Tempol failed to improve renal blood flow, cortical perfusion or GFR and the administration of an angiotensin receptor blocker (ARB) produced only a delayed and attenuated reduction in MAP. Similar results have been observed in a hypercholesterolemic pig model.

Thus, the role that increased levels of Ang II and subsequent oxidative stress play during the chronic phase of 2K,1C renovascular hypertension is not well understood. As such, there are currently no specific therapies or suggested solutions for treating chronic renal hypertension or preventing the progression of early renal/renovascular hypertension to the chronic stage.

### BRIEF SUMMARY OF THE INVENTION

Disclosed herein are methods and compositions for the treatment of chronic renal hypertension by administering a therapeutically effective amount of at least one nitroxide-containing composition to a subject. Also disclosed herein are methods and compositions for delaying the onset of renal failure in a subject comprising administering a therapeutically effective amount of at least one nitroxide-containing composition to a subject.

In particular embodiments, the nitroxide-containing composition comprises 4-hydroxy-2,2,6,6-tetramethyl-1-piperidine-1-oxyl (Tempol).

In particular embodiments, the subject is selected from the group consisting of humans, non-human primates, rabbits, rats, mice, cats, dogs, horses, and cows.

In particular embodiments, the nitroxide-containing composition is administered intravenously. In other embodiments, the nitroxide-containing composition is administered orally.

In particular embodiments, the nitroxide-containing composition is formulated for sustained delivery.

In particular embodiments, the therapeutically effective amount is from about 1 to about 700 mg/kg/day.

### BRIEF SUMMARY OF THE SEVERAL VIEWS OF THE DRAWINGS

FIG. 1 is an index of the body weights, kidney weights, baseline mean arterial blood pressure (MAP) and kidney tissue pO₂ in sham rats or rats with a silver clip placed on the renal artery of the left kidney (2K,1C) prior to the various acute interventions. The designation (a) denotes a statistically significant difference (p<0.05) when compared to corresponding sham group, and the designation (b) denotes a statistically significant difference (p<0.05) when compared to the contralateral kidney within the same group.

FIG. 2 illustrates the mean±SEM values for mean arterial blood pressure in anesthetized sham and 2K,1C rats during baseline conditions and during the different acute interventions. An asterisk (*) denotes a statistically significant difference (p<0.05) when compared to baseline with the same group, and a cross (†) denotes a statistically significant difference (p<0.05) when compared to the candesartan, enalaprilat or combined treatment groups within the same category of animals.

FIG. 3 illustrates the mean changes in renal cortical blood flow in sham and 2K,1C rats during the different acute interventions. An asterisk (*) denotes a statistically significant difference (p<0.05) when compared to baseline with the same group, and a cross (†) denotes a statistically significant difference (p<0.05) when compared to the candesartan, enalaprilat or combined treatment groups within the same category of animals.

FIG. 4 illustrates the mean changes in renal cortical oxygen tension in sham and 2K,1C rats during the different acute interventions. An asterisk (*) denotes a statistically significant difference (p<0.05) when compared to baseline with the same group, and a cross (†) denotes a statistically significant difference (p<0.05) when compared to the candesartan, enalaprilat or combined treatment groups within the same category of animals.

FIG. 5 illustrates the mean changes in renal medullary oxygen tension in sham and 2K,1C rats during the different acute interventions. An asterisk (*) denotes a statistically significant difference (p<0.05) when compared to baseline with the same group, and a cross (†) denotes a statistically significant difference (p<0.05) when compared to the candesartan, enalaprilat or combined treatment groups within the same category of animals.

FIG. 6 is an illustration of H&E (hematoxylin and eosin) and endothelin-1 (ED-1) stains of portions of non-clipped and clipped kidneys. Sections from non-clipped (A) and clipped (B) kidneys were counterstained with hematoxylin-eosin. ED-1 expression in non-clipped (C) and clipped (D) kidneys was detected using an ELISA further described in Example 1. Macrophage infiltration is noticeable in the cortex of the clipped kidneys (D). The bar in the lower right hand corner of each cell is 50 μm in length and is provided for perspective.

FIG. 7 is a Western blot analysis of interleukin-6 (IL-6; A) and NADPH oxidase p22^{phox} (B) expression in non-clipped and clipped kidneys. An asterisk (*) denotes a statistically significant difference (p<0.05) when compared to the non-clipped kidney.

### DETAILED DESCRIPTION OF THE INVENTION

Disclosed herein are methods and compositions for the treatment of chronic renal hypertension by administering at least administering a therapeutically effective amount of at least one nitroxide-containing composition to a subject. Also disclosed herein are methods and compositions for delaying the onset of renal failure in a subject comprising administering a therapeutically effective amount of at least one nitroxide-containing composition to a subject.

Nitroxides, or aminoxyl radicals, are chemical compounds having the common structure R₂N—O.<=>R₂N.⁺—O. They are low molecular weight compounds that are metal-independent, nontoxic and non-allergenic, and are characterized by low reactivity with oxygen, high solubility in aqueous solutions, and the ability to cross cellular membranes. The lipophilicity of nitroxides can be controlled by the addition of various organic substituents in order to facilitate the targeting of specific organs or organelles.

Nitroxides have been shown to protect cells and animals against the effects of free radicals and reactive oxygen species species, such as superoxide, by functioning as antioxidants. See, e.g., U.S. Pat. No. 5,462,946. Examples of nitroxides suitable for use in the present methods and compositions may be found in e.g., U.S. Patent Application Publication No. 20070021323.

A nitroxide particularly contemplated for use in the present methods and compositions is 4-hydroxy-2,2,6,6-tetramethyl-1-piperide-1-oxyl (a.k.a. Tempol). Tempol is an antioxidant and superoxide dismutase mimetic that has a demonstrated anti-hypertensive effect in essential hypertension. See e.g., U.S. Pat. Nos. 6,096,759 and 6,617,337. As solutions containing Tempol discolor when left in sunlight, it is recommended that compositions containing them be protected from light.

Surprisingly, the present inventors have discovered that the acute administration of Tempol lowers MAP, increases blood flow, increases tissue pO₂, and lowers blood pressure in the clipped kidney of chronic 2K,1C Goldblatt rats. In fact, Tempol is more effective in lowering MAP in chronic 2K,1C Goldblatt rats than blockade of the renin-angiotensin system with an ARB, AT₂-R antagonist or an ACE inhibitor. Furthermore, the inventors have unexpectedly discovered that only Tempol effectively increases both the cortical blood flow and the tissue pO₂ in the renal cortex and medulla in the clipped kidney of chronic 2K,1C Goldblatt rats, whereas interventions directed against the renin-angiotensin system had no effect. These observations all indicate that Tempol and other nitroxides are effective in reversing or attenuating symptoms of chronic renal hypertension and delaying the onset of renal failure, and are useful in methods and compositions to treat these and other related conditions.

In certain embodiments, these compositions are formulated as pharmaceutical preparations. In particular embodiments, pharmaceutical preparations are formulated as pharmaceutically-acceptable salts. Lists of suitable salts are found in, for example, Remington: The Science and Practice of Pharmacy, 21st Edition (Lippincott Williams & Wilkins, 2006).

In other embodiments, pharmaceutical preparations are formulated as pharmaceutical compositions comprising one or more of the active agents or pharmaceutically-acceptable salts thereof together with a pharmaceutically-acceptable carrier. Suitable pharmaceutical carriers are described, for example, in Remington: The Science and Practice of Pharmacy, 21st Edition (Lippincott Williams & Wilkins, 2006).

In particular embodiments, the pharmaceutically-acceptable carriers further comprise excipients and auxiliaries to facilitate processing of the active compounds into formulations for delivery to the site of action. Suitable formulations for parenteral administration include aqueous solutions of the active compounds in water-soluble form; for example, water-soluble salts. Oily injection suspensions of the active compounds may also be administered. Suitable lipophilic solvents or vehicles include fatty oils, such as sesame oil or synthetic fatty acid esters (e.g., ethyl oleate or triglycerides). Aqueous injection suspensions can contain substances that increase the viscosity of the suspension. These include, for example, sodium carboxymethyl cellulose, sorbitol, and dextran. Optionally, the suspension can also contain stabilizers.

In particular embodiments, the pharmaceutical compositions are formulated for sustained delivery of the compounds in accordance with the present methods for a period of 24 hours to a month or more. Sustained-release formulations for oral administration once a day are particularly contemplated. Such formulations are described, for example, in U.S. Pat. Nos. 5,968,895 and 6,180,608. Any pharmaceutically-acceptable, sustained-release formulation known in the art is contemplated.

As used herein, administering or administration includes dispensing, delivering or applying a compound disclosed herein in a method disclosed herein, e.g., in a pharmaceutical formulation, to a subject by any suitable route for delivery of that compound to the desired location in the subject. Intravenous administration is particularly contemplated, but oral, rectal and subcutaneous administration is contemplated as well.

Suitable formulations for oral administration include hard or soft gelatin capsules, pills, tablets, including coated tablets, elixirs, suspensions, syrups or inhalations and controlled release forms thereof.

Suitable formulations for topical administration include any common topical formulation such as a solution, suspension, gel, ointment or salve and the like can be employed. Preparations of such topical formulations are well described in the art of pharmaceutical formulations as exemplified, for example, by Remington: The Science and Practice of Pharmacy, 21st Edition (Lippincott Williams & Wilkins, 2006). For topical application, the formulations disclosed herein can be administered as a powder or spray, particularly in aerosol form.

It is contemplated that the compounds disclosed herein are administered in a therapeutically effective amount, i.e., an amount sufficient to achieve a desired result. An effective amount is also one in which any toxic or detrimental effects associated with administration of the compound are outweighed by the therapeutically beneficial effects. For example, effectiveness may be determined by e.g., blood pressure (systolic pressure, diastolic pressure, mean arterial pressure, CBF, PO₂, proteinuria, albuminuria, GFR, kidney morphology, and any other indicators of oxidative stress in the blood or the kidney.

The compounds disclosed herein, and formulations thereof, can be administered to a wide variety of subjects. Subjects include humans, non-human primates, rabbits, rats, mice, cats, dogs, horses, and cows. Among the wide variety of subjects, humans are particularly contemplated.

Effective amounts of the compounds used in the methods of the invention may vary according to factors such as the disease state, age, and weight of the subject, and the ability of the compound to elicit a desired response in the subject. Dosage regimens can be adjusted to provide the optimum therapeutic response. Accordingly, dosages for the methods disclosed herein range from about 1 mg/kg/day to 700 mg/kg/day.

### EXAMPLES

The invention is now described with reference to the following Example. These Examples are provided for the purpose of illustration only, and the invention is not limited to these Examples, but rather encompasses all variations which are evident as a result of the teachings provided herein.

### Example 1

As described in Welch et al., Hypertension, 41:692-696 (2003) and Palm et al., Hypertension, 51:345-351 (2008), young male Sprague-Dawley rats (80-100 g) were anesthetized with isoflurane (0.5-1.5%). A silver clip (0.2 mm) was placed around the left renal artery (2K,1C). Age-matched rats were used as controls (sham).

All rats received hydralazine+hydrochlorothiazide+reserpine (HHR; 30+10+0.2 mg·kg⁻¹·day⁻¹) in the drinking water as described in Welch et al., Kidney Int., 63:202-208 (2003) in order to maximize survival of the clipped rats. The HHR treatment was discontinued 14 days prior to undergoing the protocol described hereinafter.

Twelve months after clipping, all rats were anesthetized with Inactin (100 mg·kg⁻¹ i.p.; Sigma-Aldrich, St. Louis, Mo.), and an endotracheal tube was inserted for spontaneous respiration. Rats were prepared according to the protocol described in Palm et al., Hypertension, 51:345-351 (2008). Briefly, the left femoral artery was catheterized for monitoring mean arterial blood pressure (MAP) and the left femoral vein for infusion of saline (5 ml·kg bw⁻¹·h⁻¹). The left kidney was immobilized in a plastic cup while renal cortical pO₂ and cortical blood flow (CBF) were measured with O₂ microelectrodes (Unisense, Aarhus, Denmark) and laser-Doppler needle probes (Transonic Systems Inc, Ithaca, N.Y.) as described in Palm et al., Diabetologia, 46:1153-1160 (2003); Palm et al., Hypertension, 51:345-351 (2008); and Liss et al., Pflügers Arch., 434:705-711 (1997).

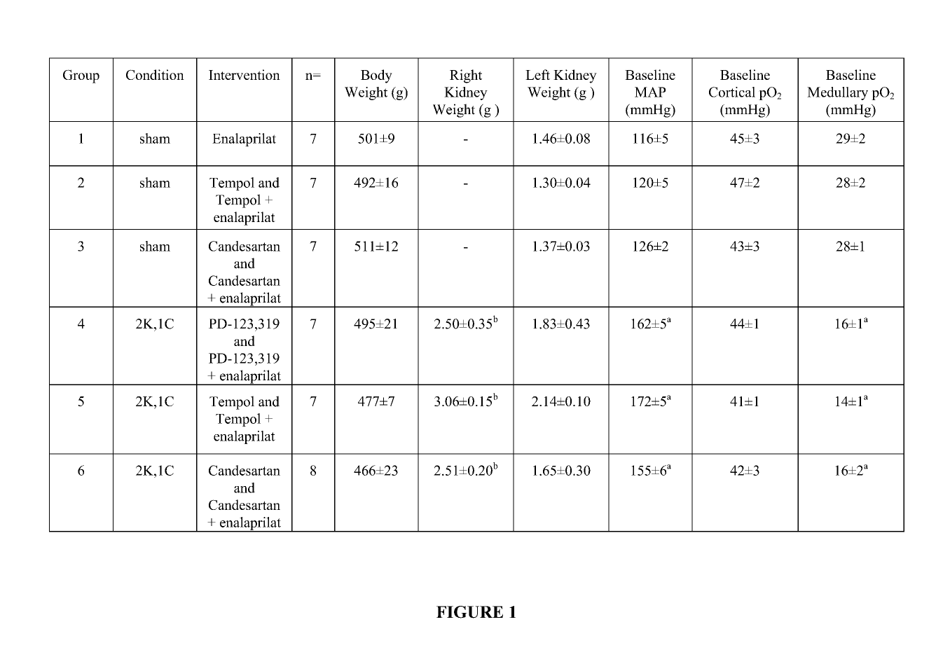

No statistically significant difference in the average body weights of sham (n=21) and 2K,1C (n=22) rats was observed (501±7 vs. 479±11 g; FIG. 1). On average, the non-clipped kidney of 2K,1C rats was significantly heavier than the non-clipped kidneys of sham rats. Contrary to what is observed in 2K,1C rats in the early stages of renal disease, the weight of the clipped kidneys of chronic 2K,1C rats did not significantly differ from their sham counterparts.

Measurements in 2K,1C rats were made before and after injections of PD-123,319 (1 mg·kg bw⁻¹ bolus+1 mg·kg bw⁻¹·h⁻¹; Sigma Aldrich) followed 30 minutes later by either enalaprilat (0.3 mg·kg bw⁻¹ bolus+0.3 mg·kg bw^{-l}·h⁻¹; Novaplus 1.25 mg·m⁻¹; Baxter Healthcare Corporation, Deerfield, Ill.) or tempol (174 μmmol·kg bw⁻¹ bolus+174 μmmol·kg bw⁻¹·h⁻¹; Sigma Aldrich) followed 30 minutes later by enalaprilat or candesartan (1 mg·kg bw⁻¹ bolus+1 mg·kg bw⁻¹·h⁻¹; provided by Astra Zeneca, Södertälje Sweden) followed after 30 minutes by enalaprilat. The same regimen was applied to sham rats with the exception that PD-123,319 was not administered since PD-123,319 has not been found to change mean arterial pressure or kidney function in normal rats. See e.g., Duke et al., Br. J. Pharmacol., 144:486-492 (2005).

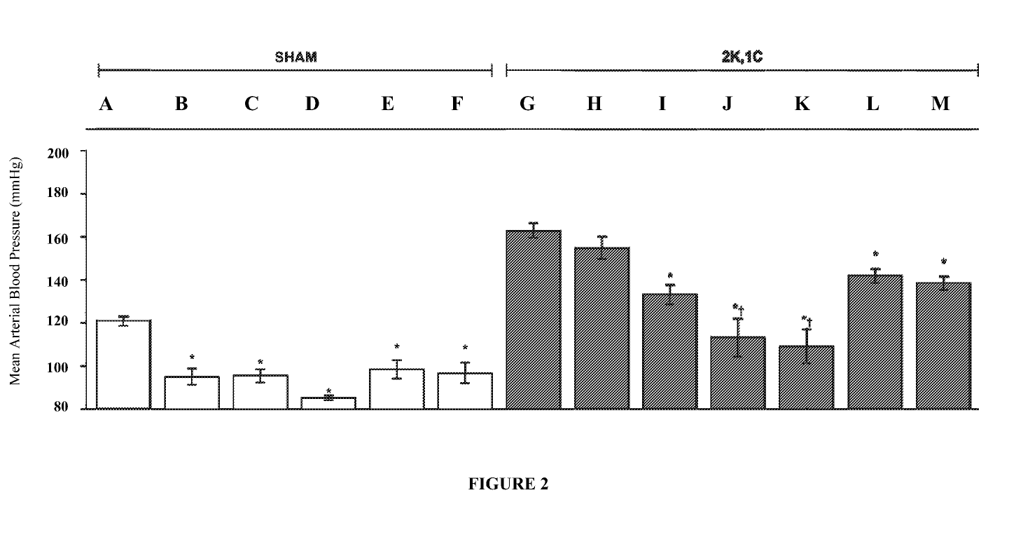

2K,1C rats had elevated MAP (FIGS. 1 and 2). All treatments reduced MAP modestly, but similarly, in elderly sham rats. The elevated MAP of 2K,1C rats was unaffected by PD-123,319 but was reduced by candesartan. However, tempol was significantly more effective (FIG. 2). Enalaprilat did not produce a further fall in MAP in rats administered tempol or candesartan, but did reduce the MAP of rats given PD-123,319.

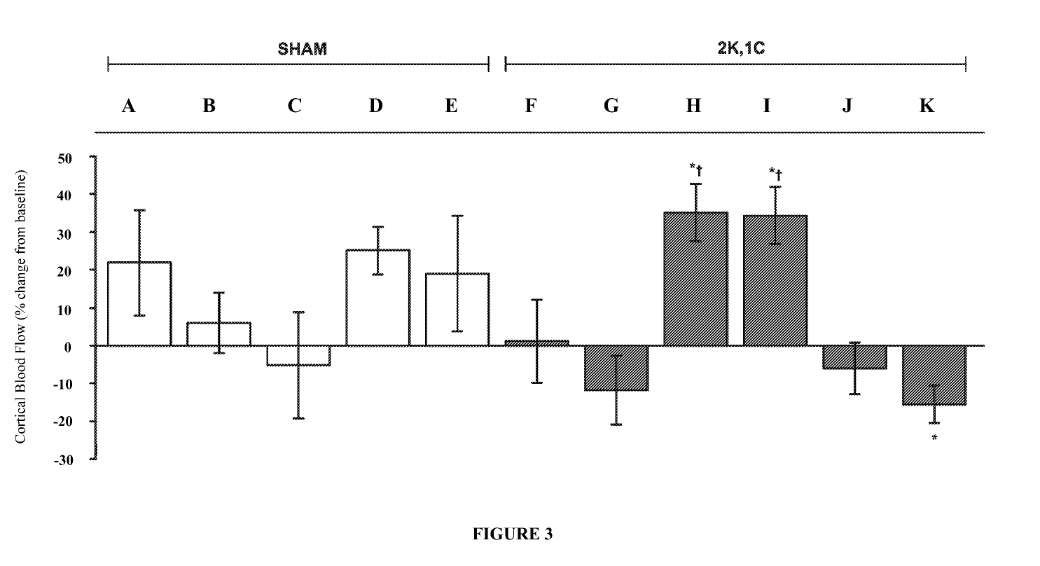

The CBF was unchanged after all applied acute interventions in sham rats. Neither candesartan nor PD-123,319 alone changed CBF significantly in 2K,1C rats (FIG. 3). However, CBF was increased in 2K,1C rats by tempol. CBF did not change further in 2K,1C rats given enalaprilat after tempol and was reduced significantly by enalaprilat following candesartan administration (FIG. 3).

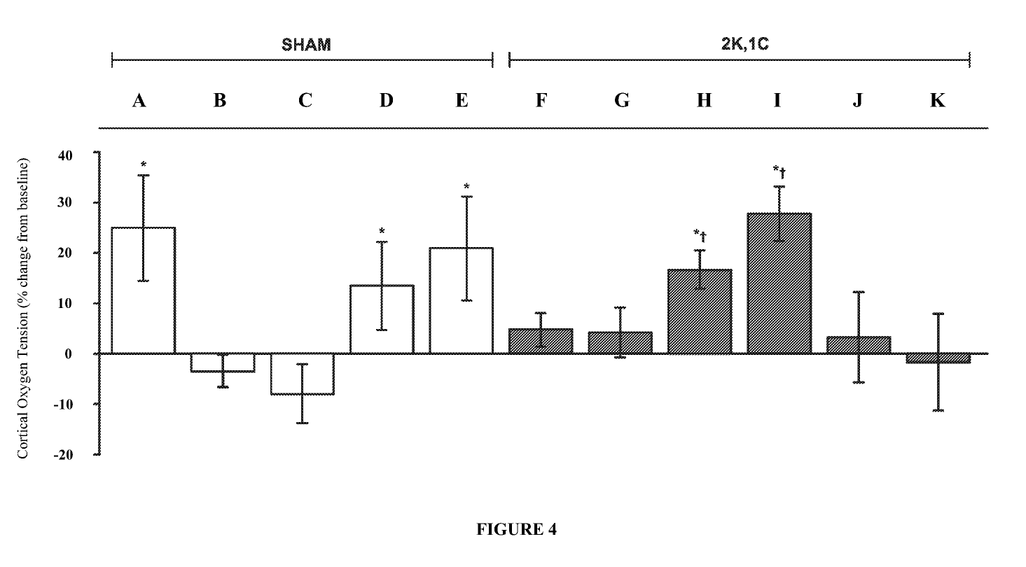

The baseline renal cortical pO₂ was similar in 2K,1C and sham rats (42±1 vs. 45±1 mmHg respectively; FIG. 1). Enalaprilat, candesartan or the combination increased cortical pO₂ in sham rats, whereas tempol was not effective in this group (FIG. 4). Only tempol increased the cortical pO₂ in 2K,1C rats (FIG. 4). This was not changed significantly when followed by enalaprilat.

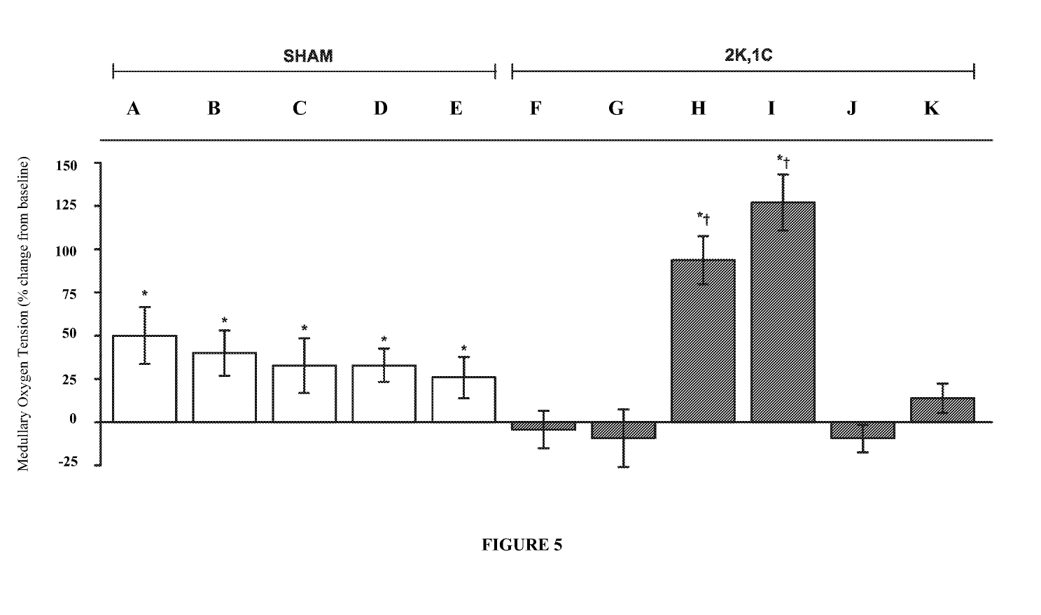

The baseline renal medullary pO₂ was reduced in 2K,1C rats compared to sham rats (16±1 vs. 28±1 mmHg; FIG. 1). Renal medullary pO₂ was increased after all the applied acute interventions in sham rats, whereas again only tempol, increased the medullary pO₂ in 2K,1C rats (FIG. 5). Subsequent administration of enalaprilat after tempol did not change the medullary pO₂ further.

Tissue samples from rat kidney cortex were homogenized in lysis buffer (1.0% NP40, 0.5% sodium deoxycholate, 0.1% SDS, 10 mM NaF, 80 mM Tris, pH 7.5) containing enzyme inhibitors (phosphatase inhibitor cocktail-2; 10 μl/ml; Sigma-Aldrich, and Complete Mini; 1 tablet/1.5 ml; Roche Diagnostics, Mannheim, Germany). 50 μg of protein was electrophoresed and blotted on to polyvinylidene fluoride membranes as described in Onozato et al., Kidney Int., 65:951-960 (2004) and Onozato et al., Kidney Int., 61:186-194 (2002). Membranes were incubated overnight with monoclonal antibodies against p22^{phox} (kindly provided by Dr. Mark Quinn) at 1:200 dilution or a polyclonal antibody for interlukin-6 (IL-6, Abcam, Cambridge, UK) at a 1:250 dilution. The membranes were subsequently incubated with horseradish peroxidase (HRP)-conjugated secondary antibody (Dako, Glostrup, Denmark) at 1:1000 dilution. HRP labeling was detected with 3,3′-diaminobenzidine (DAB; Sigma-Aldrich). Western blot bands were quantified and analyzed with NIH ImageJ software.

Frozen tissue sections were stained with hematoxylin-eosin. Kidney slices were processed for immunohistochemistry using the labeled streptavidin biotin method, as described in Onozato et al., Kidney Int., 65:951-960 (2004) and Onozato et al., Kidney Int., 61:186-194 (2002).

Additional frozen tissue sections (5 μm) were incubated with monoclonal antibody recognizing ED-1 (BMA Biomedicals AG, August, Switzerland) at 1:100 dilution, followed by incubation with biotinylated anti-rabbit IgG secondary antibody (Dako) at a 1:400 dilution, and then with HRP-conjugated streptavidin solution (Dako). HRP labeling was detected using a peroxide substrate solution containing DAB.

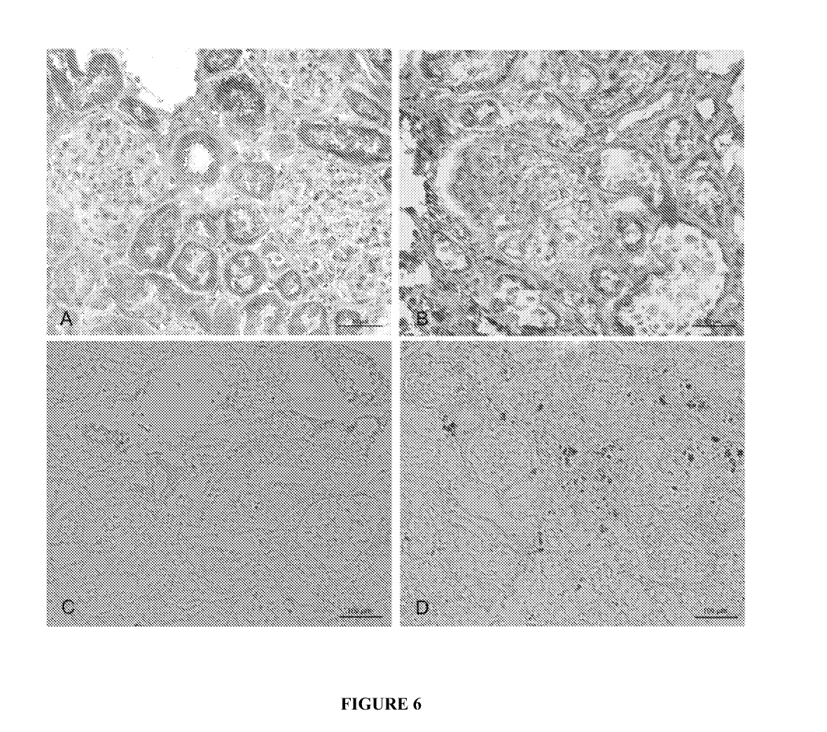

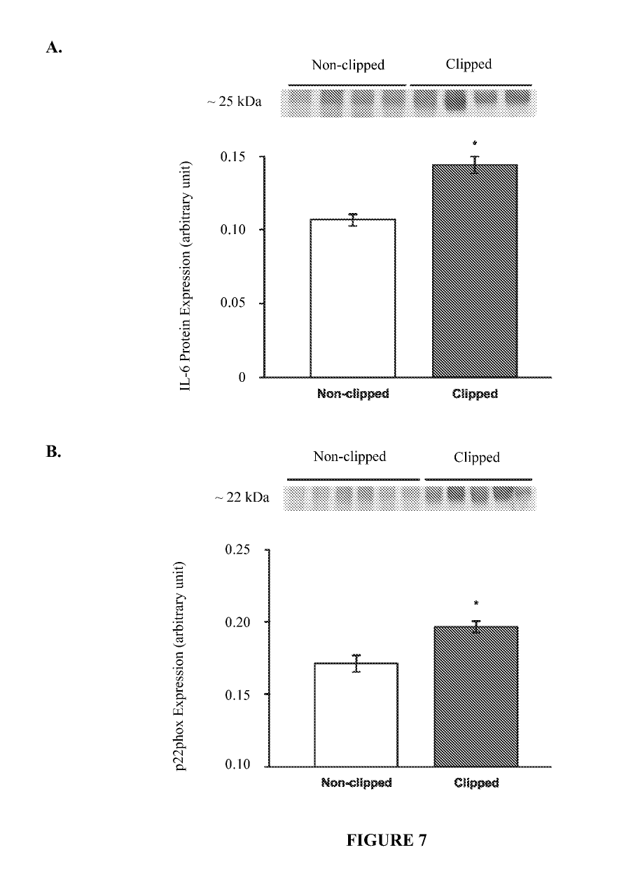

The non-clipped kidney showed normal morphology features (FIG. 6A) while glomeruli in the clipped kidney showed segmental sclerosis lesions (FIG. 6B). Macrophage infiltration, detected by immunostaining with its maker ED-1, was increased significantly in the cortex of clipped compared non-clipped kidneys (FIG. 6). Macrophages can produce the inflammatory cytokine interleukin-6 (IL-6), which was indeed increased in the clipped, compared to the non-clipped, kidney (FIG. 7A). Furthermore, the NADPH oxidase subunit p22^{phox} was elevated significantly in the clipped, compared to the non-clipped, kidney (FIG. 7B).

All procedures described in Example 1 were performed under guidelines recommended by the National Institutes of Health and approved by the Georgetown University Animal Care and Use Committee.

The data in Example 1 was analyzed using ANOVA for multiple data sets and Student's t-test for paired comparisons. When appropriate, this was followed by Dunnett's post hoc. Relative changes displayed in FIGS. 1-4 are for visualization purposes only; statistics were calculated using the original parametric data sets (GraphPad Prism, GraphPad Software, San Diego Calif.). The Mann-Whitney test was used for Western blot densitometry data. For all comparisons, p<0.05 was considered statistically significant. All values are expressed as mean±SEM.

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

What is claimed is:

1. A method of treating chronic renal hypertension in a subject comprising administering a therapeutically effective amount of at least one nitroxide-containing composition to the subject.

2. The method of claim 1, wherein the nitroxide-containing composition comprises 4-hydroxy-2,2,6,6-tetramethyl-1-piperidine-1-oxyl (Tempol).

3. The method of claim 1, wherein the nitroxide-containing composition is administered intravenously.

4. The method of claim 1, wherein the wherein the nitroxide-containing composition is administered orally.

5. The method of claim 4, wherein the nitroxide-containing composition is formulated for sustained delivery.

6. The method of claim 2, wherein the therapeutically effective amount of 4-hydroxy-2,2,6,6-tetramethyl-1-piperidine-1-oxyl (Tempol) is from about 1 to about 700 mg/kg/day.

7. The method of claim 6, wherein the nitroxide-containing composition is administered intravenously.

8. The method of claim 6, wherein the wherein the nitroxide-containing composition is administered orally.

9. The method of claim 8, wherein the nitroxide-containing composition is formulated for sustained delivery.

10. A method of delaying the onset of renal failure in a subject comprising administering a therapeutically effective amount of at least one nitroxide-containing composition to the subject.

11. The method of claim 10, wherein the nitroxide-containing composition comprises 4-hydroxy-2,2,6,6-tetramethyl-1-piperidine-1-oxyl (Tempol).

12. The method of claim 10, wherein the nitroxide-containing composition is administered intravenously.

13. The method of claim 10, wherein the wherein the nitroxide-containing composition is administered orally.

14. The method of claim 13, wherein the nitroxide-containing composition is formulated for sustained delivery.

15. The method of claim 11, wherein the therapeutically effective amount of 4-hydroxy-2,2,6,6-tetramethyl-1-piperidine-1-oxyl (Tempol) is from about 1 to about 700 mg/kg/day.

16. The method of claim 15, wherein the nitroxide-containing composition is administered intravenously.

17. The method of claim 15, wherein the wherein the nitroxide-containing composition is administered orally.

18. The method of claim 17, wherein the nitroxide-containing composition is formulated for sustained delivery.
